(19) United States
(12) Patent Application Publication
(10) Pub. No.: US 2026/0345707 A1
(43) Pub. Date: Nov. 5, 2026

(54) ANTIALLERGIC AGENT

(71) Applicant: SONE FARM CO., LTD., Tokyo (JP)

(72) Inventor: Masanori SUGIYAMA, Hiroshima-Shi (JP)

(73) Assignee: SONE FARM CO., LTD., Tokyo (JP)

(21) Appl. No.: 19/335,419

(22) Filed: Sep. 22, 2025

(30) Foreign Application Priority Data
Apr. 30, 2025 (JP) 2025-075372

(51) Int. Cl. A61K 36/284; A61P 37/08

(52) U.S. Cl. CPC A61K 36/284; A61P 37/08; A61K 2236/19; A61K 2236/331; A61K 2236/37

(57) ABSTRACT

The objective is to provide a composition that is effective in treating or preventing food allergies and has few side effects.

The present invention relates to a composition for preventing or improving the symptoms of food allergies, which contains a fermented product obtained by fermenting a Byakujutsu extract liquid with lactic acid bacteria.

### TECHNICAL FIELD

The present invention relates to a composition for preventing or improving food allergies, which contains as an active ingredient a fermented product of Atractylodes Japonica Rhizome, and in particular to a composition for preventing or improving food allergies, which contains as an active ingredient a fermented product obtained by fermenting an extract of Atractylodes Japonica Rhizome with lactic acid bacteria Lacticacillus eibacillus paracasei.

### BACKGROUND TECHNOLOGY

In recent years, the number of food allergy sufferers has been increasing, which has become a social problem. Wheat is one of the grains that can cause severe allergic reactions, and the number of people with wheat allergies has been increasing over the past 10 years. It is known that the symptoms of wheat allergy differ between adults and children, with children more likely to develop atopic eczema and dermatitis syndrome (AEDS) and adults more likely to develop wheat-dependent exercise-induced anaphylaxis (WDEIA). It is also known that a compound called @-5 gliadin contained in gluten, an insoluble protein derived from wheat, is the main causative allergen of WDEIA.

Up to now, methods have been proposed for treating food allergies by administering to a subject a composition containing Anaerostipes caccae and prebiotics, or a composition containing two or more purified bacterial strains (Patent Documents 1 and 2). In addition, a method for creating a wheat variety that does not have the @-5 gliadin gene has been proposed (Patent Document 3).

Byakujutsu (White Atractylodes) is herbal medicine made from the dried rhizomes of Atractylodes japonica Koidzumi, a member of the Asteraceae family. It has been reported to have a wide range of pharmacological effects against various diseases, including improving gastrointestinal function, immunomodulatory effects, anti-inflammatory effects, antibacterial effects, and antitumor effects. In addition, Byakujutsu is known to contain multiple anti-inflammatory components, some of which have been reported to suppress systemic and passive cutaneous anaphylactic reactions in model mice (Non-patent Reference 1), but the beneficial effects of Byakujutsu on food allergies remain unclear and further research is needed.

Medicinal herbs contain substances that are expected to have valuable therapeutic effects, and in general, the intestinal microflora can biotransform these plant-derived substances into bioactive substances that exert therapeutic effects. This means that gut bacteria play a key role in maximizing the efficacy of these herbs by biotransforming complex substances into more compatible components through fermentation. This process improves the bioavailability and activity of phytochemicals, leading to a significant increase in functional microbial metabolites, which in turn have beneficial effects on human health. In fact, previous studies have found that fermented herbal extracts fermented by plant-derived lactic acid bacteria contain bioactive molecules (Non-Patent Document 2).

### PRIOR ART DOCUMENTS

### Patent Documents

[Patent Document 1] Japanese Patent Publication No. 2022-510780

[Patent Document 2] Japanese Patent Publication No. 2023-121755

[Patent Document 3] Japanese Patent Publication No. 2020-110094

### Non-Patent Documents

[Non-patent document 1] J. Agric. Food Chem. 2016, 64, 2254-2262.

[Non-patent document 2] Antioxidants 2021, 10, 1071.

### SUMMARY OF THE INVENTION

### Problem to be Solved by the Invention

Herbal medicines are generally considered to have fewer side effects than Western medicines, making them easier to incorporate into treatment. The object of the present invention is to provide a composition that is effective in treating or preventing food allergies and has few side effects, focusing on the specific effects of Byakujutsu and fermented extract.

### Means for Solving the Problem

The inventors discovered that a composition containing a fermented product obtained by fermenting with plant-derived lactic acid bacteria reduces the symptoms of food allergies, particularly wheat allergies, and completed the present invention.

That is, the present invention is summarized as follows.
[1] A composition for preventing or improving food allergies, comprising a fermented product obtained by fermenting a Byakujutsu extract liquid with lactic acid bacteria.
[2] The composition according to [1], in which the lactic acid bacteria is Lacticaseibacillus paracasei.
[3] The composition according to [2], wherein the lactic acid bacterium is Lacticaseibacillus paracasei IJH-SONE68 (accession number: NITE BP-02242).
[4] The composition according to [1], in which the allergen of food allergies is wheat.
[5] The composition according to [1], which is a composition that reduces anaphylactic reactions.
[6] The composition according to [1], which is a composition that reduces the IgE concentration in serum.
[7] The composition according to [1], which is a composition that increases the IFN-γ concentration in serum.
[8] The composition according to [1], which is a composition that reduces the IL-4 concentration in serum.
[9] A food or drink for food allergies, containing the composition according to [1].
[10] A medicine for food allergies, containing the composition according to [1].
[11] A method for preventing or treating food allergies, comprising a step of administering the medicine for food allergies according to [10].

### Effects of the Invention

According to the present invention, a composition that can reduce the symptoms of food allergies, particularly wheat allergies, can be provided.

### BRIEF DESCRIPTION OF THE DRAWINGS

FIG. 1 A graph of the anaphylaxis scores of a model mouse.

FIG. 2 A graph of the IgE concentration in serum of a model mouse.

FIG. 3 Graphs of IFN-y concentration (A) and IL-4 concentration (B) in serum of a model mouse.

### EMBODIMENT FOR CARRYING OUT THE INVENTION

The wheat allergy composition of the present invention will be specifically described hereinafter.

“Byakujutsu” in the present invention includes the herbal medicine “Wabyakujutsu” made from the dried rhizome of a plant called Okera (Atractylodes japonica Koidzumi) and the herbal medicine “Karabyakujutsu” made from the dried rhizome of a plant called Oobanaokera (Atractylodes macrocephala Koidzumi), with “Wabyakujutsu” being preferably used. In addition, “Byakujutsu” is a herbal medicine different from “Sojutsu”, which is the rhizome of Atractylodes lancea De Candolle or Atractylodes chinensis Koidzumi.

The “Byakujutsu extract liquid” in the present invention can be an extract liquid obtained by extracting Byakujutsu using any extraction method known in the art. For example, a room temperature extraction or heat extraction method using an extraction solvent such as distilled water, alcohols such as methanol, or a mixture of these can be used, and extraction can be performed under reduced pressure or pressure if necessary, but is not limited to these methods. In addition, supercritical extraction or subcritical extraction using carbon dioxide, etc., extraction using distillation such as steam distillation, extraction by squeezing, reflux extraction, etc. can also be used. A method of heat extraction under pressure using water as a solvent is preferred in terms of extraction efficiency and cost. Dried Byakujutsu is added and mixed with water to a concentration of 5~10% (w/v), and then heated to 100~130 °C under pressure for 30~60 minutes to perform extraction. The extracted liquid can be treated by any known method, such as centrifugation, sterilization, or filtration. As the Byakujutsu extract liquid, a concentrate of Byakujutsu extract liquid or a solution obtained by drying the concentrate and dissolving the concentrate in water can be used. Commercially available extracts, concentrates, and dried powders can also be used.

The “fermented product” in the present invention is a liquid obtained by fermenting Byakujutsu extract liquid with lactic acid bacteria. Fermenting Byakujutsu extract liquid improves the bioavailability and activity of the active ingredients contained in the extract liquid, thereby improving the anti-allergic effect of the composition.

The method of fermentation with lactic acid bacteria is not particularly limited, but may include a method of adding a seed culture solution of lactic acid bacteria to a Byakujutsu extract liquid and culturing the lactic acid bacteria in the extract liquid to carry out fermentation. The amount of lactic acid bacteria culture solution added may be, for example, 0.05~3 v/v % relative to the Byakujutsu extract liquid. The culture conditions are not particularly limited, but it is preferable to culture at 20~30°C for 1~5 days. The fermented liquid after fermentation can be treated by any known method such as centrifugation, sterilization, or filtration.

The “lactic acid bacteria” in the present invention is not particularly limited as long as it does not interfere with the effects of the present invention, but Lactobacillus lactic acid bacteria can be used. Plant-derived lactic acid bacteria can grow more actively in plant extract liquids than animal-derived lactic acid bacteria, so that it is preferable to use plant-derived Lactobacillus lactic acid bacteria. More preferably, it is plant-derived Lacticaseibacillus paracasei, and most preferably Lacticaseibacillus paracasei IJH-SONE68 derived from fig leaves. The above-mentioned Lacticaseibacillus paracasei IJH-SONE68 derived from fig leaves was deposited on Apr. 19, 2016 (original deposit date) at the National Institute of Technology and Evaluation, Patent Microorganism Depositary Center (Japan: 2-5-8 Kazusa Kamashichi, Kisarazu City, Chiba Prefecture 292-0818) as NITE P-02242. Furthermore, the above strain was transferred from the original deposit to a deposit based on the Budapest Treaty on May 19, 2017 and deposited as NITE BP-02242. In addition, Lacticaseibacillus paracasei was previously named Lactobacillus paracasei, but it was reclassified and its scientific name changed based on taxonomic verification based on genome analysis in 2020. The above strain was deposited under its old scientific name. “Food allergy” in the present invention is a phenomenon in which adverse symptoms are induced in the living body through an antigen-specific immunological mechanism caused by food, and food allergy appears when a specific food is ingested or touched. Food allergies can cause a variety of symptoms, including skin symptoms such as redness, hives, eczema, and edema; respiratory symptoms such as coughing, wheezing, and shortness of breath; mucosal symptoms such as bloodshot eyes, tears, runny nose, and swelling of the lips and tongue; digestive symptoms such as nausea, vomiting, abdominal pain, and diarrhea; neurological symptoms such as headache, loss of consciousness, and incontinence; and circulatory symptoms such as low blood pressure, tachycardia, and cyanosis. The severity of these symptoms varies greatly from person to person. Anaphylaxis is defined as a hypersensitivity reaction that can be life-threatening (rather than a minor symptom) in which systemic allergic symptoms, including those affecting multiple organs, are induced. Foods that cause allergies can include a variety of foods, such as wheat, chicken eggs, milk, nuts, fruits, fish, fish eggs, buckwheat, and soybeans, but the composition of the present invention is particularly effective in preventing or improving wheat allergy.

The “food and drink for food allergies” in the present invention may be any food and drink containing the composition of the present invention, and may be added to the raw materials during the manufacturing process of the food and drink. Examples of food and drink include dairy products such as cheese, fermented milk, lactic acid bacteria drinks, butter, margarine, etc., milk drinks, fruit juice drinks, soft drinks, etc., egg products such as jelly, candy, pudding, mayonnaise, etc., and sweets and breads such as butter cake, but are not limited to these food and drink.

The “medicine for food allergies” in the present invention is a medicine that uses the composition of the present invention as a raw material, and can be in any dosage form such as tablets, capsules, powder, syrup, etc. In addition, when preparing the formulation, it can be formulated by appropriately mixing excipients, stabilizers, flavorings, etc. that are permitted for formulation, or it can be used as a powder or dusting agent by drying the composition of the present invention as it is. In addition, the formulation may be prepared by mixing excipients, binders, disintegrants, lubricants, flavorings, suspending agents, coating agents, and any other optional agents, as long as they do not interfere with the food allergy prevention/amelioration effect.

### EXAMPLES

The present invention will be described in more detail below with reference to examples, but the present invention is not limited to these examples.

### Preparation of Byakujutsu Extract Liquid

The rhizomes of A. japonica were obtained in a dried and cut state from Kojima Kampo Co., Ltd. The rhizome pieces were mixed with distilled water to a final concentration of 7.5% (w/v), and heated at 105 °C for 30 minutes under pressure. The solid residue was removed by centrifugation, and the pH of the resulting supernatant was adjusted to 7.0 with NaOH solution, and then sterilized at 120 °C for 15 minutes to produce Byakujutsu extract liquid.

### Preparation of Lactic Acid Bacteria Culture Solution

Fig leaf-derived lactic acid bacteria L. paracasei IJH-SONE 68 (accession number: NITE BP-02242) was inoculated into MRS Broth (Merck) and statically cultured at 28 °C to prepare a lactic acid bacteria culture solution.

### Preparation of Fermented Byakujutsu Extract Liquid

The lactic acid bacteria culture solution was added to Byakujutsu extract liquid at a ratio of 1% (v/v) and incubated at 28 °C for 24 hours. After fermentation, the culture solution was sterilized at 120 °C for 20 minutes and the bacterial residue was removed by centrifugation. The resulting supernatant was used as the fermented Byakujutsu extract liquid.

### Preparation of Gliadin

50 g of flour (Nissin Flour Milling Co., Ltd.) was mixed with 10 times the amount of 0.5 M sodium chloride solution and stirred at room temperature for 2 hours to extract salt-soluble proteins. After centrifugation, 500 mL of distilled water was added to the residue and stirred at room temperature for 2 hours to extract water-soluble albumin and salt into the water, and the residue was centrifuged and collected. The same extraction procedure was repeated twice, and then 500 mL of 70% (v/v) ethanol was added to the residue and stirred at 4 °C for 2 hours to extract gliadin, which was then centrifuged to obtain an ethanol fraction. The ethanol fraction was injected into a dialysis membrane and dialyzed against a 1% (v/v) acetic acid solution at 4 °C for 60 hours. During dialysis, the acetic acid solution was replaced every 2~3 hours on the first day, and 2~ times a day from the second day onwards. The dialysate was then freeze-dried to obtain gliadin.

### Mouse Experiments

Seven-week-old female SPF BALB/cJ mice were obtained from Shimizu Experimental Materials Co., Ltd. Mice were housed in plastic cages in a controlled environment with a temperature of 20 ~26 °C, humidity of 40~0%, and a 12-hour light-dark cycle. Mice were freely fed MF rodent diet (Oriental Yeast Co., Ltd.) ad libitum and had unlimited access to drinking water. After one week of acclimation, mice were randomly divided into four experimental groups of five mice each: a non-sensitized group (negative control, NC group), a sensitized untreated group (sensitized positive control, PC group), a sensitized unfermented Byakujutsu extract-treated group (Unf-AJR group), and a sensitized fermented Byakujutsu extract-treated group (Fer-AJR group).

To induce allergic symptoms, mice in the PC, Unf-AJR, and Fer-AJR groups were orally administered 100 μL of 10 mg/mL gliadin solution on days 3, 7, and 11. Gliadin was dissolved in a linoleic acid/lecithin (4:1) mixture containing 0.75% (v/v) ethanol and 3 mg/mL sodium salicylate.

During the experimental period, mice in the Unf-AJR and Fer-AJR groups were orally administered 100 μL of AJR extract (unfermented or fermented) every day, and mice in the NC and PC groups were administered the same amount of sterile water. On day 14, mice in the PC, Unf-AJR, and Fer-AJR groups were administered 100 times the amount of gliadin.

On the final day of the experiment (day 15), the mice were euthanized and blood samples were taken. Serum IgE, IFN-γ, and IL-4 concentrations were measured using enzyme-linked immunosorbent assay (ELISA) kits (BioLegend). Mice were scored for anaphylaxis using the following scoring system: asymptomatic individuals were scored as 0, scratching of the mouth or ears with the hind paws or ruffling of the fur was scored as 1, nausea was scored as 2, difficulty in breathing or cyanosis of the mouth or tail was scored as 3, and severe symptoms leading to death within 24 hours were scored as 4. GraphPad Prism 8 software (GraphPad Software, Inc.) was used for all statistical analyses. Tukey-Kramer tests were used for multiple comparison analyses. When not applicable, the Steel-Dwass test was used instead.

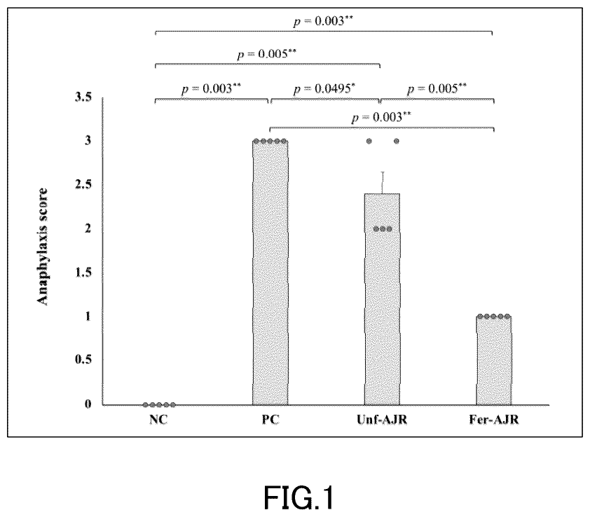

FIG. 1 shows the results of measuring the anaphylaxis scores of mice. After gliadin administration, the PC and Unf-AJR groups showed significant anaphylaxis. In contrast, the Fer-AJR group showed only mild symptoms. The anaphylaxis scores of the Fer-AJR group were significantly different from those of the PC group (p=0.003).

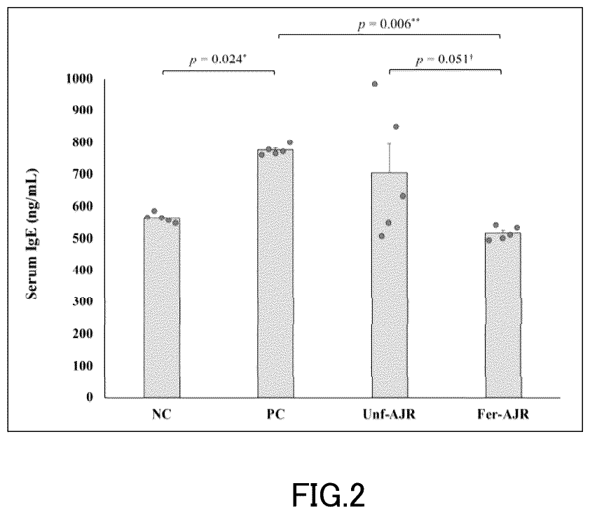

It is known that the allergic reaction observed in gliadin-induced anaphylaxis is mediated by IgE and involves IgE-dependent decomposition of mast cells. FIG. 2 shows the results of measuring the IgE concentration in mouse serum. The mean IgE concentration of the PC group was significantly higher than that of the NC and Fer-AJR groups (p=0.024 and p=0.006, respectively), reflecting the allergic symptoms. The Unf-AJR group showed no improvement in IgE concentration.

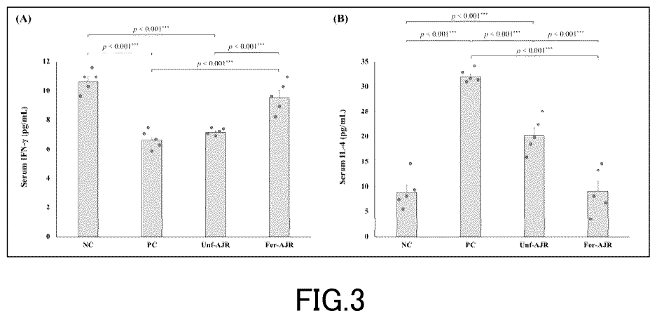

Generally, food allergies are caused by an imbalance in the Th1/Th2 response ratio, so that an excessive Th2 immune response can be used to evaluate allergic symptoms. To confirm changes in the Th1/Th2 balance, serum concentrations of IFN-γ, a Th1 cytokine, and IL-4, a Th2 cytokine, were measured, and the results are shown in FIG. 3 (IFN-γ concentration (A), IL-4 concentration (B)).

Compared to the NC group, the PC group mice showed a significant decrease and increase in the values of IFN-γ (p<0.001) and IL-4 (p<0.001), respectively. These cytokine values were almost restored in the Fer-AJR group, and the secretion balance was normalized, but only a slight improvement in the value of IL-4 was observed in the Unf-AJR group.

1. A composition for preventing or improving food allergies, comprising a fermented product obtained by fermenting a Byakujutsu extract liquid with lactic acid bacteria.

2. The composition according to claim 1, in which the lactic acid bacteria is Lacticaseibacillus paracasei.

3. The composition according to claim2, wherein the lactic acid bacterium is Lacticaseibacillus paracasei IJH-SONE68 (accession number: NITE BP-02242).

4. The composition according to claim 1, in which the allergen of food allergy is wheat.

5. The composition according to claim 1, which is a composition that reduces anaphylactic reactions.

6. The composition according to claim 1, which is a composition that reduces the IgE concentration in serum.

7. The composition according to claim 1, which is a composition that increases the IFN-γ concentration in serum.

8. The composition according to claim 1, which is a composition that reduces the IL-4 concentration in serum.

9. A food or drink for food allergies, comprising the composition according to claim 1.

10. A medicine for food allergies, comprising the composition according to claim 1.

11. A method for preventing or treating food allergies, comprising a step of administering the medicine for food allergies according to claim 10.
